(19) United States
(12) Patent Application Publication
(10) Pub. No.: US 2013/0224122 A1
(43) Pub. Date: Aug. 29, 2013

(54) MAGNETOTATIC BACTERIA MRI POSITIVE CONTRACT ENHANCEMENT AGENT AND METHODS OF USE

(71) Applicant: The Board of Trustees of the Leland Stanford Junior University, (US)

(72) Inventors: Sanjiv S. Gambhir, Portola Valley, CA (US); Michael Benoit, Menlo Park, CA (US); A. C. Matin, Stanford, CA (US); Yoram Barak, Menlo Park, CA (US); Shay Keren, Haifa (IL); Dirk Mayer, Menlo Park, CA (US)

(73) Assignee: The Board of Trustees of the Leland Stanford Junior University, Palo Alto, CA (US)

(21) Appl. No.: 13/845,923

(22) Filed: Mar. 18, 2013

Related U.S. Application Data
(62) Division of application No. 12/627,299, filed on Nov. 30, 2009.
(60) Provisional application No. 61/200,683, filed on Dec. 2, 2008.

(51) Int. Cl. A61K 49/18

(52) U.S. Cl. CPC A61K 49/1896
USPC 424/9.32

(57) ABSTRACT

Magnetic resonance imaging (MRI) is enhanced by contrast agents such as superparamagnetic iron-oxide (SPIO) particles that resemble magnetite particles produced by magnetotactic bacteria. Magnetospirillum magneticum AMB-1 produces positive MRI contrast when generating ultrasmall magnetite particles (10-40 nm diameter). Positive MRI contrast permits clearer distinction from image voids compared to negative contrast. T1-weighted MRI showed that such bacteria increased positive contrast 2.2-fold (p<0.02) in vitro and 2.0-fold (p<0.02) following intratumoral injection in mouse tumor xenografts. Upon intravenous delivery, Magnetospirillum magneticum AMB-1 targeted tumors and generated increased positive MRI contrast in them (1.4-fold; p<0.01). AMB-1 tumor targeting was shown by viable counts, microPET imaging of radio-labeled AMB-1, and Prussian blue staining of tumor sections. Thus, magnetotactic bacteria provide a tool for improving cancer diagnosis and monitoring treatment response by MRI.

### CROSS-REFERENCE TO RELATED APPLICATIONS

This application claims priority to U.S. Provisional Patent Application Ser. No. 61/200,683 entitled “MAGNETOTACTIC BACTERIA MRI POSITIVE CONTRAST ENHANCEMENT AGENT AND METHODS OF USE” filed on Dec. 2, 2008, the entirety of which is hereby incorporated by reference.

### STATEMENT REGARDING FEDERALLY SPONSORED RESEARCH OR DEVELOPMENT

This disclosure was made with government support under NIH Grant Nos. NIH/NCI P50 CA114747, R01 CA125074-01A1, RR09784, NIH/NIGMS F32GM077827, and T32-AI07328 awarded by the U.S. National Institutes of Health of the United States government. The government has certain rights in the disclosure.

### TECHNICAL FIELD

The present disclosure is generally related to the cultivation of magnetotactic bacteria for use as magnetic resonance imaging positive contrast enhancement agents. The disclosure further relates to methods of detecting tumors in a subject by magnetotactic bacterial enhancement of the positive contrast of magnetic resonance images.

### BACKGROUND

Magnetic resonance imaging (MRI) is a routine diagnostic tool for anatomical imaging. Its advantages over other imaging techniques include superior (sub-millimeter) spatial resolution, lack of radiation burden, and unlimited tissue penetration. To enhance its sensitivity, contrast agents such as small paramagnetic iron oxide (SPIO) particles may be used. Paramagnetic agents can enhance both positive (bright) and negative (dark) contrast by locally altering the magnetic field (Thorek et al., (2006) Ann. Biomed. Eng. 34: 23-38). The effect is to shorten relaxation of nuclear spins following radio frequency perturbation. Relaxation times in the longitudinal and transverse planes of the magnetic field are referred to as T1 and T2, respectively. Weighting the MRI parameters for T1 enhances positive contrast, while T2-weighting enhances negative contrast. Positive contrast is often preferable for anatomical imaging.

Targeting of contrast agents to specific tissues can enhance MRI usefulness in diagnosis and cellular tracking. One approach toward this end is to incorporate SPIO particles into mammalian cells that target certain tissues, such as tumors, which can then be tracked with MRI. However, multiplication of the SPIO-bearing cells in the target tissue will decrease the amount of SPIO per cell, which limits the efficacy of this approach (Rogers et al., (2006) Nat. Clin. Pract. Cardiovasc. Med. 3: 554-562). A genetically-encoded contrast agent could overcome this limitation by producing new agent in situ.

SPIO particles resemble magnetite (Fe₃O₄) particles produced by magnetotactic bacteria (Bazylinski & Frankel (2004) Nat. Rev. Microbiol. 2: 217-230; Blakemore R. P. (1975) Science 190: 3787-3793). These bacteria live in aquatic environments and use magnetite to align themselves along the Earth's geomagnetic field, enabling them to find the low-oxygen conditions they require for growth (Smith et al., (2006) Biophys. J. 91: 1098-1107). In addition, many bacteria, especially anaerobes (e.g., Clostridia sp. (Brown & Wilson (2004) Nat. Rev. Cancer. 4: 437-447; Dang et al., (2001) Proc. Natl. Acad. Sci. U.S.A. 98: 15155-15160; Liu et al., (2002) Gene Ther. 9: 291-296)) and facultative anaerobes (e.g., Salmonella sp. (Kasinskas & Forbes (2007) Cancer Res. 67: 3201-3209; Loessner et al., (2007) Cell Microbiol. 9: 1529-1537; Soghomonyan et al. (2005) Cancer Gene Ther. 12: 101-108; Zhao et al., (2007) Proc. Natl. Acad. Sci. U.S.A. 104: 10170-10174; Zhao et al., (2006) Cancer Res. 66: 7647-7652), specifically target tumors.

Recently, it was shown that mammalian cells encoding a gene from magnetotactic bacteria can enhance MRI negative contrast (Zurkiya et al., (2008) Magn. Reson. Med. 59: 1225-1231). Negative contrast, however, which is the reduction in an MRI signaling has the potential to be confused with other areas of a tissue that are not highlighted by MRI, whereas positive contrasting that increases the highlight makes detection of the signal more apparent to the observer.

### SUMMARY

Briefly described, embodiments of this disclosure encompass, among others, magnetotactic bacterial agents with the ability to target tumors and provide improved visualization thereof using enhanced-positive contrast magnetic resonance imaging (MRI). The disclosure takes advantage of magnetotactic bacteria that naturally produce magnetite (Fe₃O₄) particles. These particles resemble super paramagnetic iron oxide (SPIO) particles that are currently used as MRI contrast agents. The size of the bacterial magnetite particles has been manipulated to make them smaller than those found in nature, which alters their MRI contrast enhancement properties. The manipulated bacteria of the present disclosure produce positive (bright) contrast, as opposed to the negative (dark) contrast that is typical of the currently available SPIO particles. The bacterial magnetite particles of the disclosure, therefore, more closely resemble ultrasmall SPIO (USPIO) particles that are known to enhance positive contrast for improved images derived by MRI.

The magnetotactic bacteria of the present disclosure are able to selectively colonize tumors in an animal subject while being cleared from normal tissue. The bacteria, therefore, may colonize tumors and enhance MRI positive contrast, thereby increasing the likelihood of tumor detection by MRI. MRI itself is a technique that provides advantages over other imaging modalities in terms of superior spatial resolution and unlimited tissue penetration. The magnetotactic bacterial imaging agents of the present disclosure, therefore, provides enhanced tumor visualization using MRI. The disclosure also provides for improved detection of cancerous tumors at an earlier stage, important for successful treatment.

One aspect of the present disclosure, therefore, provides methods of cultivating a magnetotactic bacterium, comprising: obtaining an isolated strain of magnetotactic bacteria capable of forming magnetite; and cultivating the magnetotactic bacteria in a growth medium comprising an iron salt, whereupon the cultivated magnetotactic bacteria synthesize magnetite particles having a diametric size between about 5 nm to about 50 nm, and where the cultivated magnetotactic bacteria are characterized as providing contrast enhancement of an magnetic resonance image of a cancerous lesion when contacted with said lesion.

In some embodiments of this aspect of the disclosure, the magnetotactic bacterium can be a strain of Magnetospirillum magneticum. In certain of these embodiments, the magnetotactic bacterium can be Magnetospirillum magneticum AMB-1 (ATCC Accession No. 700264).

Another aspect of the present disclosure provides a bacterial population cultivated in a growth medium comprising an iron salt, whereupon the cultivated bacteria comprises magnetite particles having a diametric size between about 5 nm to about 50 nm, and where the bacteria population is characterized as providing contrast enhancement of an magnetic resonance image of a cancerous lesion when contacted with said lesion.

Another aspect of the disclosure provides compositions comprising magnetotactic bacteria cultivated in a growth medium comprising an iron salt, whereupon the cultivated bacteria comprise magnetite particles having a diametric size between about 5 nm to about 50 nm; and a pharmaceutically acceptable carrier, and where the cultivated magnetotactic bacteria are characterized as providing contrast enhancement of an magnetic resonance image of a cancerous lesion when contacted with said lesion.

Yet another aspect of the disclosure provides methods of obtaining enhancement of positive contrast of a magnetic resonance image, comprising: delivering to a subject an amount of a composition comprising magnetotactic bacteria obtained by cultivating the magnetotactic bacteria in a growth medium comprising an iron salt, whereupon the cultivated bacteria comprise magnetite particles having a diametric size between about 5 nm to about 50 nm and where the cultivated magnetotactic bacteria are characterized as providing contrast enhancement of an magnetic resonance image of a cancerous lesion when contacted with said lesion; and a pharmaceutically acceptable carrier; allowing the magnetotactic bacteria to selectively target a tissue of the subject; and obtaining a magnetic resonance image of the subject, wherein the magnetotactic bacteria provide a magnetic resonance image having enhanced positive contrast.

Still yet another aspect of the disclosure provides methods of detecting a target tissue in a subject, comprising: delivering to a subject an amount of a composition comprising magnetotactic Magnetospirillum magneticum AMB-1 (ATCC Accession No. 700264) bacteria obtained by cultivating the magnetotactic bacteria in a growth medium comprising ferric chloride, whereupon the cultivated bacteria comprise magnetite particles having a diametric size between about 15 nm to about 30 nm, where the cultivated magnetotactic bacteria are characterized as providing contrast enhancement of an magnetic resonance image of a cancerous lesion when contacted with said lesion; and a pharmaceutically acceptable carrier, wherein the composition is delivered to a tissue of the subject, the tissue; and obtaining a magnetic resonance image of the subject, where the magnetotactic bacteria provide a magnetic resonance image having enhanced positive contrast, thereby detecting the tissue of the subject.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1A shows digital images of axial-slice images for Magnetospirillum magneticum AMB-1 with low magnetite content (FIG. 1A, upper panels) or high magnetite content (FIG. 1A, lower panels) suspended in 3% gelatin at increasing cell concentrations.

FIG. 1B shows a graph of mean (±1 s.d.) MRI intensities for Magnetospirillum magneticum AMB-1 having low or high magnetite content (p<0.05 at every cell concentration).

FIG. 1C is a graph illustrating magnetic moment (proportional to the quantity of magnetite) for Magnetospirillum magneticum AMB-1 having low or high magnetite content.

FIGS. 2A-2G show digital transmission electron microscope images of Magnetospirillum magneticum AMB-1 with low magnetite content (FIGS. 2A-2C), or high magnetite content (FIGS. 2D-2F) showing the smaller size of magnetite particles in FIGS. 2A-2C versus those shown in FIGS. 2D-2F. Each scale bar indicates 100 nm; arrows indicate some of the small magnetite particles).

FIG. 2G shows a histogram graphically illustrating the particle diameter distribution for low (white bars) or high (black bars) magnetite content. The frequency is the fraction of the total number of particles measured (>100 particles per group). The data are grouped into 5 nm bins.

FIGS. 3A-3D show the correlation between Magnetospirillum magneticum AMB-1 cell number and T1-weighted positive contrast in mouse tumors injected directly with Magnetospirillum magneticum AMB-1.

FIG. 3A is a graph illustrating the normalized signal intensities from T1-weighted MR images of mouse tumors injected intratumorally with increasing concentrations of AMB-1 cells. Immediately post-injection, the number of bacterial cells in tumors is the same as the number injected because the injected bacteria remain localized to the tumor for several hours.

FIGS. 3B-3D show a series of T1-weighted axial-slice images of a tumor pre-injection (FIG. 3B), immediately post-injection (FIG. 3C) and 1 day post-injection (FIG. 3D) with 3.75×10⁸ AMB-1 cells. The gradient maps highlight the location of the intratumoral injections; the corresponding scale bar illustrates the normalized signal intensity.

FIGS. 4A-4C is a series of digital axial-slice images showing enhanced MRI contrast in tumors (highlighted with gradient maps) after intratumoral delivery of Magnetospirillum magneticum AMB-1 (right tumor) but not in the control tumor (left), immediately post-injection (FIG. 4A), 1 day later (FIG. 4B), and 6 days later (FIG. 4C). The color bar shows the normalized signal intensity within the color gradient maps.

FIG. 4D is a graph showing the relative tumor signal intensities from four mice (each tumor was normalized by its contralateral control); bars indicate mean±1 s.d. for control (white bars) or test tumors (black bars).

FIG. 4E shows a series of digital photomicrographs where iron and bacterial staining indicate that magnetotactic bacteria remain in tumors for 7 days. 400× magnification images of tumor sections stained with Prussian blue for iron (Panel A, left); Gram stain for bacteria (Panel B, clumps of small cells arrowed); highlighted section from Panel B enlarged to show gram negative bacteria (small panel C); 1000× magnification black-and-white image from the same section as Panel C showing individual bacteria (black spots).

FIG. 5 is a graph showing the number of colony forming units (CFU) per gram of tissue recovered from the tumor, liver, and spleen of mice (n=3) after 1, 3, and 6 days following tail-vein injection with 1×10⁹ Magnetospirillum magneticum AMB-1 cells.

FIG. 6A shows a pair of digital MR images series illustrating that magnetotactic bacteria produce positive contrast in tumor xenografts following systemic delivery. In each series (upper and lower) the T1-weighted axial-slice MR images are of a mouse tumor prior to injection (Image A), 2 days post-injection (Image B), and 6 days post-injection (via tail-vein with 1×10⁹ bacteria suspended in 100 μl MSGM) (Image C). The grey bar shows normalized signal intensity within the gradient maps. In the lower series of images, tumors are indicated by arrows, and the MR images are shown without overlays.

FIG. 6B is a graph illustrating that the signal increased 1.22-fold (* p=0.003) after 2 days and 1.39-fold (** p=0.0007) after 6 days (n=4).

FIG. 7A shows digital decay-corrected, coronal-slice microPET images of mice at indicated times (h) after intravenous delivery of ⁶⁴Cu-PTSM-labeled Magnetospirillum magneticum AMB-1 (FIG. 7A, upper), or ⁶⁴Cu-PTSM alone (FIG. 7A, lower). The grey bar represents the percentage of the injected dose of ⁶⁴Cu activity per gram of tissue (% ID/g); the arrows indicate tumor locations. An outline of the mouse is traced in the 16 h images for anatomical reference.

FIG. 7B is a graph illustrating the mean (+1 s.d.) signal intensity in tumors showed an increase due to AMB-1 that was not observed in the control group.

FIG. 7C is a graph illustrating that in normal tissue (liver and spleen) the mean (+1 SD) signal peaked by 4 hours, from both ⁶⁴Cu-labeled bacteria (AMB-1) and ⁶⁴Cu-PTSM (control) groups. Phagocytosis by spleen macrophages can account for the high % ID/g in spleen of the magnetotactic bacterial group compared to the control group at early time points.

FIG. 7D is a digital decay-corrected coronal-slice images at three times post-injection (arrows point to the tumor location; an outline of the mouse is traced in the 16 h image for anatomical reference) showing that for 64Cu-PTSM labeled Magnetospirillum magneticum AMB-1 delivered intratumorally, the bacteria largely remain in the tumor.

FIG. 7E is a graph showing the mean % ID/g (+1 s.d.) at times after injection for the tumor, liver and spleen.

FIG. 8A is a digital image, T2-weighted and showing negative contrast of axial-slice images for Magnetospirillum magneticum AMB-1 with magnetite particles of about 50 nm or greater suspended in 3% gelatin at increasing cell concentrations.

FIG. 8B shows a graph of mean (±1 s.d.) intensities for the negative contrast images Magnetospirillum magneticum AMB-1 with magnetite particles of about 50 nm or greater at different cell concentrations (p<0.05 at every cell concentration).

The drawings are described in greater detail in the description and examples below.

### DETAILED DESCRIPTION

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a cell” includes a plurality or multiplicity of cells. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

Prior to describing the various embodiments, the following definitions are provided and should be used unless otherwise indicated.

### Abbreviations

MRI, magnetic resonance imaging; PET, positron emission tomography; SPIO, super paramagnetic iron oxide; MSGM, Magnetospirillum growth medium.

### Definitions

In describing and claiming the disclosed subject matter, the following terminology will be used in accordance with the definitions set forth below.

Unless otherwise defined, all technical and scientific terms used herein have the same meaning as commonly understood by one of ordinary skill in the art of molecular biology. Although methods and materials similar or equivalent to those described herein can be used in the practice or testing of the present disclosure, suitable methods and materials are described herein.

Further definitions are provided in context below. Unless otherwise defined, all technical and scientific terms used herein have the same meaning as commonly understood by one of ordinary skill in the art of molecular biology. Although methods and materials similar or equivalent to those described herein can be used in the practice or testing of the present disclosure, suitable methods and materials are described herein.

The term “magnetotactic bacterium” as used herein refers to a class of bacteria discovered in the 1970s that exhibit the ability to orient themselves along the magnetic field lines of Earth's magnetic field. Such bacteria include, but are not limited to, Magnetospirillum gryphiswaldense MSR-1, Magnetospirillum magneticum AMB-1, Magnetospirillum magnetotacticum MS-1, and the like. Although not intended to be limiting, of particular use in the embodiments of the disclosure is the strain Magnetospirillum magneticum AMB-1. The term magnetotaxis has been coined to describe the biological phenomenon upon which these microorganisms tend to move in response to the magnetic characteristics of the environment.

The term “cultivating” as used herein refers to the maintenance of a culture of a bacterial population in a viable state and allowing the proliferation of said bacteria. As used herein, the term refers to allowing the proliferation of a strain of magnetotactic bacteria in or on a culture medium comprising an iron salt that allows the generation of a predictable size of magnetite particle in the bacterial cells.

The term “magnetite” as used herein refers to a ferromagnetic mineral with chemical formula Fe₃O₄, one of several iron oxides and a member of the spinel group. The chemical IUPAC name is iron(II, III) oxide and the common chemical name ferrous-ferric oxide. The formula for magnetite may also be written as FeO.Fe₂O₃, which is one part wustite (FeO) and one part hematite (Fe₂O₃). This refers to the different oxidation states of the iron in one structure, not a solid solution.

The term “iron salt” as used herein refers to an inorganic or organic salt of a ferrous or ferric ion. The term iron salt may include, but is not limited to, iron malate, iron oxalate, iron succinate, iron citrate, iron chloride, iron sulfate, and iron nitrate, and the like.

The term “pharmaceutically acceptable carrier” as used herein includes any and all solvents, dispersion media, coatings, antibacterial and antifungal agents, isotonic agents, absorption delaying agents, and the like. The formulations or compositions of the present disclosure may also contain stabilizers, preservatives, buffers, antioxidants, or other additives known to those of skill in the art. The use of such media and agents for pharmaceutically active substances is well known in the art. Supplementary active compounds can also be incorporated into the imaging agent of the disclosure. The imaging agent of the disclosure may further be administered to an individual in an appropriate diluent or adjuvant, co-administered with enzyme inhibitors or in an appropriate carrier such as human serum albumin or liposomes. Pharmaceutically acceptable diluents include sterile saline and other aqueous buffer solutions. Adjuvants contemplated herein include resorcinols, non-ionic surfactants such as polyoxyethylene oleyl ether and n-hexadecyl polyethylene ether. Enzyme inhibitors may include pancreatic trypsin inhibitor, diethyl pyrocarbonate, trasylol, and the like.

The term “magnetic resonance imaging (MRI)” as used herein refers to a medical imaging technique most commonly used in radiology to visualize the structure and function of the body. It provides detailed images of the body in any plane. MRI uses no ionizing radiation, but uses a powerful magnetic field to align the nuclear magnetization of (usually) hydrogen atoms in water in the body. Radiofrequency fields are used to systematically alter the alignment of this magnetization, causing the hydrogen nuclei to produce a rotating magnetic field detectable by the scanner. This signal can be manipulated by additional magnetic fields to build up enough information to construct an image of the body. When a subject lies in a scanner, the hydrogen nuclei (i.e., protons) found in abundance in an animal body in water molecules, align with the strong main magnetic field. A second electromagnetic field that oscillates at radiofrequencies and is perpendicular to the main field, is then pulsed to push a proportion of the protons out of alignment with the main field. These protons then drift back into alignment with the main field, emitting a detectable radiofrequency signal as they do so. Since protons in different tissues of the body (e.g., fat versus muscle) realign at different speeds, the different structures of the body can be revealed. Contrast agents may be injected intravenously to enhance the appearance of blood vessels, tumors or inflammation. MRI is used to image every part of the body, but is particularly useful in neurological conditions, disorders of the muscles and joints, for evaluating tumors and showing abnormalities in the heart and blood vessels.

The term “positive contrast” as used herein refers to the differences in the observed MRI image between that of a targeted tissue site that generates a greater detectable signal intensity than the intensity of a signal generated in a surrounding tissue.

The term “negative contrast” as used herein refers to the difference in the observed MRI image between that of a targeted tissue site that has a lower detectable signal intensity than the intensity of a signal generated in a surrounding tissue.

The term “subject” as used herein refers to any animal, including a human, to which a composition according to the disclosure may be delivered or administered.

The term “cancer”, as used herein shall be given its ordinary meaning and is a general term for diseases in which abnormal cells divide without control. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor (a term that may further include a “cancerous lesion”) is formed. Generally, a solid tumor is an abnormal mass of tissue that usually does not contain cysts or liquid areas (some brain tumors do have cysts and central necrotic areas filled with liquid). A single tumor may even have different populations of cells within it with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, bladder cancer, breast cancer, colorectal cancer, endometrial cancer, head & neck cancer, leukemia, lung cancer, lymphoma, melanoma, non-small-cell lung cancer, ovarian cancer, prostate cancer, testicular cancer, uterine cancer, cervical cancer.

Cardiovascular disease, as used herein, shall be given its ordinary meaning, and includes, but is not limited to, high blood pressure, diabetes, coronary artery disease, valvular heart disease, congenital heart disease, arrthymia, cardiomyopathy, CHF, atherosclerosis, inflamed or unstable plaque associated conditions, restinosis, infarction, thromboses, post-operative coagulative disorders, and stroke.

Inflammatory disease, as used herein, shall be given its ordinary meaning, and can include, but is not limited to, autoimmune diseases such as arthritis, rheumatoid arthritis, multiple sclerosis, systemic lupus erythematosus, other diseases such as asthma, psoriasis, inflammatory bowel syndrome, neurological degenerative diseases such as Alzheimer's disease, Parkinson's disease, Huntington's disease, vascular dementia, and other pathological conditions such as epilepsy, migraines, stroke and trauma.

### Description

The magnetotactic bacteria and methods of the present disclosure produce greater positive contrast magnetic resonance images when compared to existing SPIO particles such as FERIDEX™. Improved positive contrast is highly desirable for MRI. Currently used or available contrast agents, such as gadolinium-based compounds, produce higher positive contrast, but they can be toxic and cannot be targeted to specific tissues. Existing SPIO contrast agents, such as FERIDEX™, do not possess targeting capabilities. Mammalian cells that do have target cell specificity and preferentially bind to tumor cells have been injected with SPIO particles and delivered in vivo as imaging enhancement agents. However, as these mammalian cells proliferate they do not generate additional SPIO particles which become progressively more diluted at each cell division.

While bacterial strains have been used to target tumors and express optical or PET based reporter genes, they have not been applied to MRI technology that provides inherently superior spatial resolution over both of these imaging modalities. Furthermore, optical-based methods have limited tissue penetration, making them ill-suited for human use. Finally, PET based methods deliver an ionizing radiation burden to the patient, which is not the case with MRI.

The magnetotactic bacteria of the present disclosure, however, can specifically target and colonize tumor tissue, while being cleared from normal tissue, and are benign agents that offer little risk, including of adventitious infection, to the recipient. The present disclosure, therefore, encompasses methods of cultivating isolated strains of bacteria, and in particular strains of the microorganism Magnetospirillum magneticum, in a manner that results in the cells having a plurality of magnetite particles that are smaller in size than those found in bacterial cells isolated from the natural environment. The methods of the present disclosure provide ferric chloride as the iron source, and cause the cells to preferentially synthesize magnetite particles in the size range of about 15 nm to about 30 nm, somewhat smaller than magnetite particles produced when the cells are cultured on media with, for example, ferric malate as the iron source. The disclosure then provides methods for the use of the Magnetospirillum magneticum having the reduced size particles to enhance positive contrast in magnetic resonance images. In particular, the MRI methods of the disclosure advantageously use the ability of the Magnetospirillum magneticum cells to selectively target localized tumors, and become concentrated therein. This concentration effect, combined with the improved properties of the magnetite particles as contrast enhancers, provides enhanced images of tumors that can increase the possibility of detecting small tumors before they may become fatal to the subject animal or human.

It is contemplated that the manipulated magnetotactic bacterial MRI-enhancing compositions of the disclosure may be delivered to the subject animal or human as a live culture, or as an inert (dead) culture. The bacterial species of the disclosure optimally grow at a temperature of about 30° C., which is below that of typical mammalian body temperatures such as 37° C. of the human species, thereby having an inherently restricted growth in a mammalian body. Selective localization within a tumor can also limit the potential for a bacterial composition to colonize the recipient subject.

While not wishing to be bound by any one theory, the bacteria also prefer hypoxic (low oxygen) conditions, that are present in solid tumors, and which may further limit potential for a bacterial composition to colonize the recipient subject. It is considered, however, within the scope of the disclosure, for the bacterial cells to be rendered inert before delivery to the recipient, thereby avoiding the possibility of a prolonged infection of a subject. Methods of providing killed bacterial cells for such as vaccines are known in the art and would be applicable to the compositions of the present disclosure.

It is further contemplated that the methods of the present disclosure may be applied to any bacterial strain that has the ability to synthesize ultrasmall magnetic particles that provide enhanced MRI imaging, and especially combined with target selectivity. The methods of the disclosure, therefore, provide a means, by adjusting the iron content of the culture medium, and most especially of the type of iron salt used, of forcing the bacterial cells to limit the size of the particles.

Many bacteria, especially anaerobes, specifically target tumors (e.g., Clostridia sp. (Brown & Wilson (2004) Nat. Rev. Cancer. 4: 437-447; Dang et al., (2001) Proc. Natl. Acad. Sci. U.S.A. 98: 15155-15160; Liu et al., (2002) Gene Ther. 9: 291-296)) and facultative anaerobes (e.g., Salmonella sp. (Kasinskas & Forbes (2007) Cancer Res. 67: 3201-3209; Loessner et al., (2007) Cell Microbiol. 9: 1529-1537; Soghomonyan et al. (2005). Cancer Gene Ther. 12: 101-108; Zhao et al., (2007) Proc. Natl. Acad. Sci. U.S.A. 104: 10170-10174; Zhao et al., (2006). Cancer Res. 66: 7647-7652). The data of the present disclosure show that tumor targeting and enhanced-contrast MRI can be realized with magnetotactic bacterium such as Magnetospirillum magneticum strain AMB-1. It is contemplated, however, that other similar strains of bacteria may be used in the compositions and methods of the disclosure, providing they are amenable to manipulation of the magnetite particles they produce by adjustments to the cultivation medium. Preferably, the bacteria will also exhibit selective targeting of a tissue or tumor that will further improve the quality of the MRI images obtained, and the diagnostic predictions derived therefrom.

### Positive MRI Contrast Generation by Magnetospirillum Magneticum AMB-1 Cells.

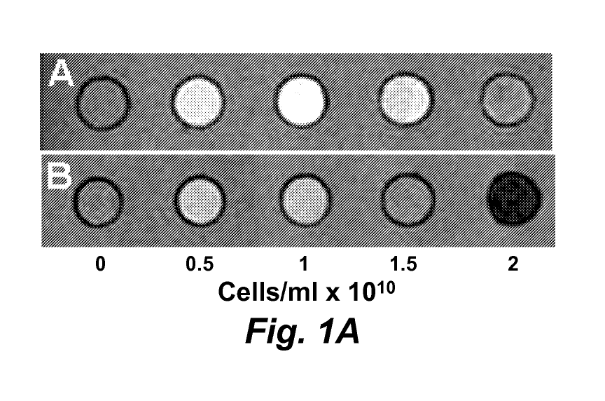

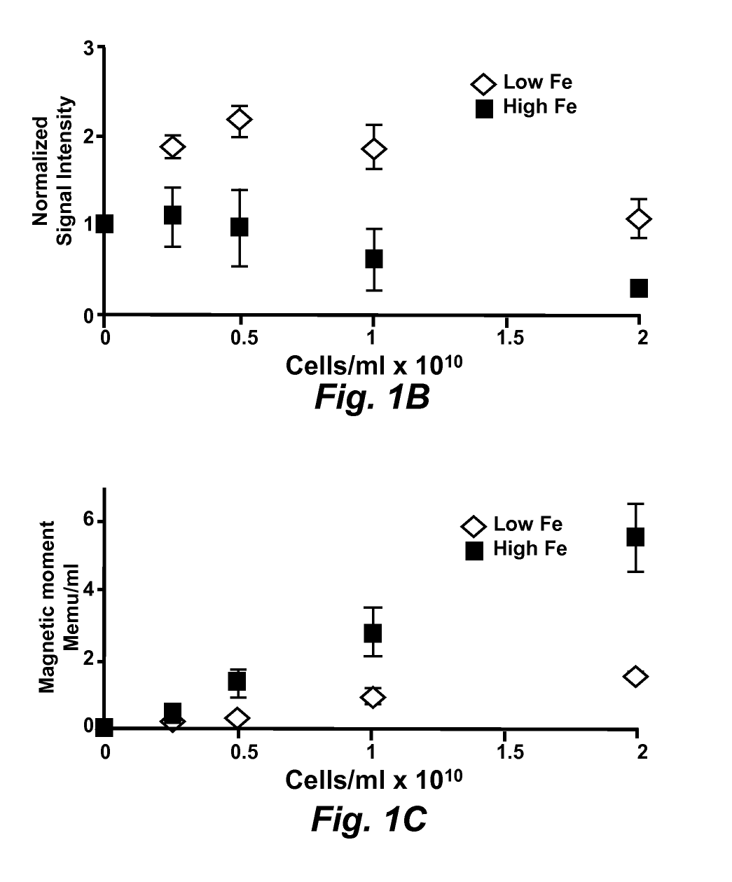

Magnetospirillum magneticum AMB-1 cells grown in MSGM medium supplemented with ferric malate generated only minimal T1-weighted positive contrast, as shown in FIG. 1A, upper panels. However, if iron in this medium is provided as ferric chloride alone, the bacteria produced significant enhanced positive contrast, as shown in FIG. 1A, lower panels. The positive contrast was seen at a cell concentration of 0.25×10¹⁰ cells/ml, and became more intense at 0.5×10¹⁰ cells/ml, but was reduced by the competing T2-effect at higher concentrations (2×10¹⁰ cells/ml).

T1-weighted contrast is quantitatively shown in arbitrary signal intensity units in FIG. 1B for different concentrations of Magnetospirillum magneticum AMB-1 grown with each iron supplement. While not wishing to be bound by any one theory, since ferric malate enhances iron availability in MSGM medium, the difference in enhanced positive contrast may have been related to total bacterial iron content. Indeed, cells grown in ferric chloride medium had a lower iron content of about 0.5×10⁻¹⁵ g/bacterium to about 0.8×10⁻¹⁵ g/bacterium (more typically about 0.64±0.08×10⁻¹⁵ g/bacterium) compared to those grown in ferric malate medium (average of 2.2±0.5×10⁻¹⁵ g/bacterium), as determined by magnetic moment measurements, as shown in FIG. 1C. These cells are herein referred to as ‘low-Fe’ and ‘high-Fe’, respectively. The low-Fe cells also caused T2-weighted signal loss (FIG. 1A, lower panel), that permits their being suitable for use as either a positive or negative contrast agent.

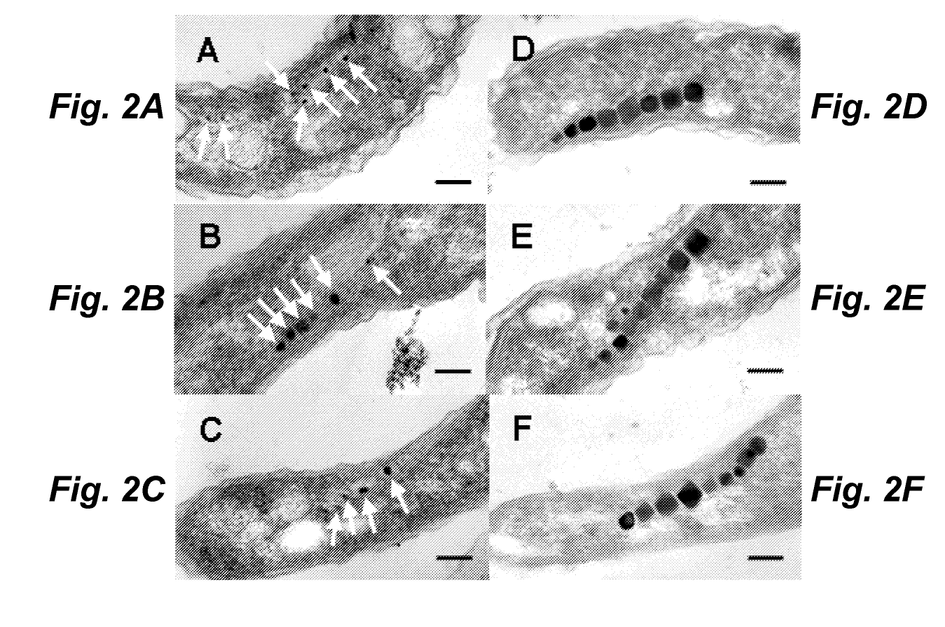

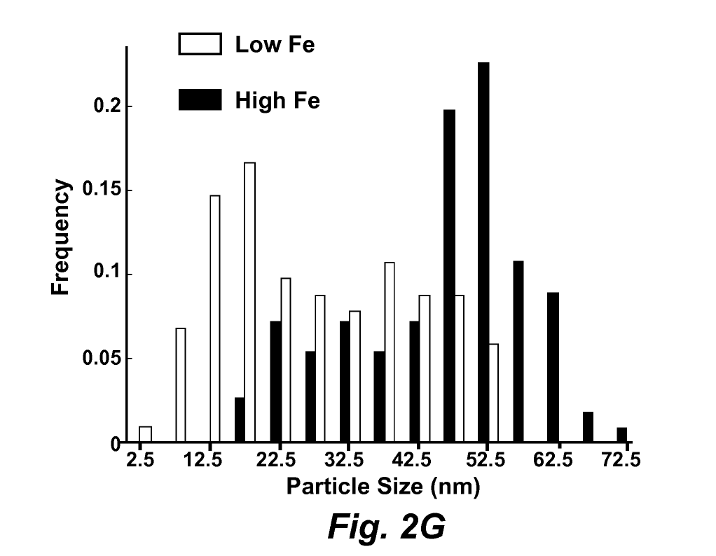

Transmission electron microscopy images showed that the low-Fe bacteria generated smaller magnetite particles compared to the high-Fe bacteria, as shown in FIGS. 2A-2F. The median particle diameters under the two conditions were 25.3 and 48.9 nm respectively, and the distribution of particle diameters (shown in FIG. 2G) was significantly different (p<2×10⁻⁶, Mann-Whitney significance test). The mean number of magnetite particles per bacterium was apparently less in low-Fe bacteria (as shown in FIGS. 2A-2C), but the difference was not significant (6.0 versus. 7.4 particles; p=0.14, Mann-Whitney significance test).

A contrast agent producing a positive signal has relatively high r₁ and low r₂ relaxivities, and exhibits a small r₂:r₁ ratio, as described by Kellar et al., (2000) J. Magn. Reson. Imaging. 11: 488-494. To further characterize the effect of Magnetospirillum magneticum AMB-1 magnetite particle size on MRI signal properties, the r₁ and r₂ of Magnetospirillum magneticum AMB-1 cells with high or low-Fe content were measured, as shown in Table 1.

TABLE 1
Relaxivites (r1 and r2) of iron oxide particles.

| Particle type | r1 (mM⁻¹s⁻¹) | r2 (mM⁻¹s⁻¹) | r2/r1 |
|---|---|---|---|
| AMB-1 low mag. | 9.3 | 337 | 36.2 |
| AMB-1 high mag | 0.68 | 48 | 70.6 |
| FERIDEXTM | 2.7 | 253 | 93.7 |

The r₂/r₁ ratio of low-Fe AMB-1 was smaller than high-Fe AMB-1. The r₂/r₁ ratio of low-Fe Magnetospirillum magneticum AMB-1 cells was also lower than that of FERIDEX™ (r2/r1=93.7), a currently used SPIO contrast agent.

### Magnetospirillum Magneticum AMB-1 Cells Produce Positive Contrast in Mouse Tumors.

To determine if the Magnetospirillum magneticum AMB-1 cells can generate positive contrast also in vivo, low-Fe AMB-1 cells were injected intratumorally in mice implanted with 293T tumor xenografts. This was done in two groups of four mice.

The first group of mice was injected with a range of Magnetospirillum magneticum AMB-1 cell concentrations (about 0.25×10¹⁰ cells/ml to about 1.0×10¹⁰ cells/ml in an injected volume of 50 μl) to determine an appropriate number of cells.

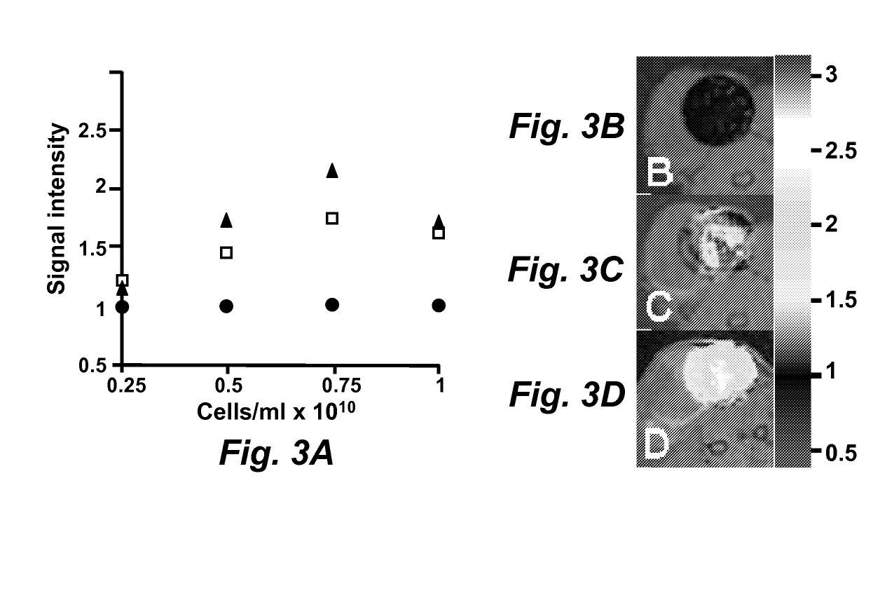

T1-weighted MR images showed increased positive contrast compared to pre-injection images for tumors injected with more than 0.25×10¹⁰ cells/ml. The positive MRI contrast generated by these bacteria in vivo (FIGS. 3A-3D) closely resembled the experimental in vitro result, shown in FIGS. 1A-1D, except that a higher number of bacteria were required in vivo. The increased signal was evident immediately following bacterial injection as well as one day later.

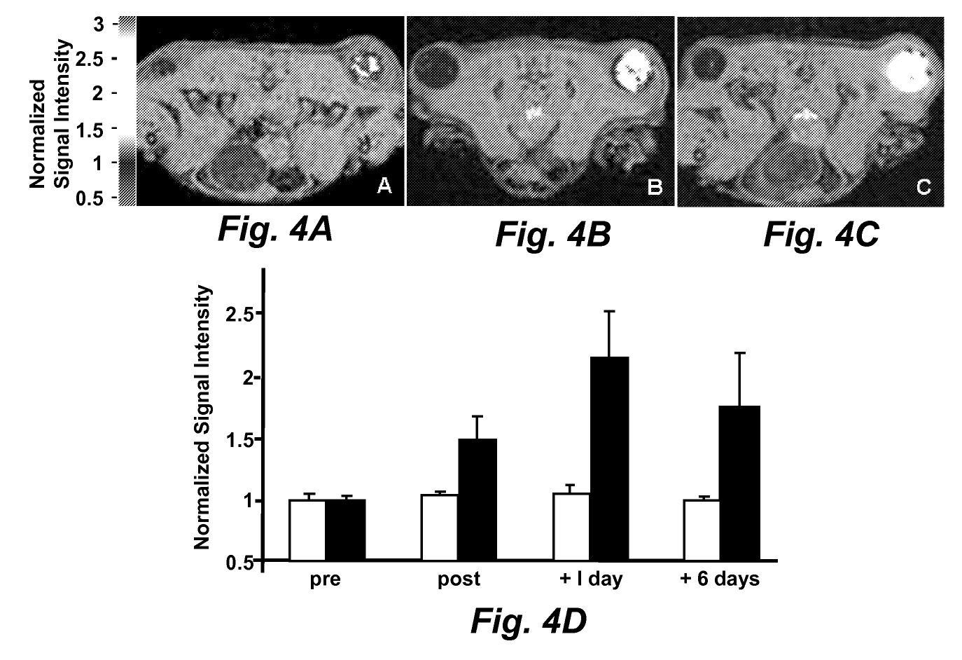

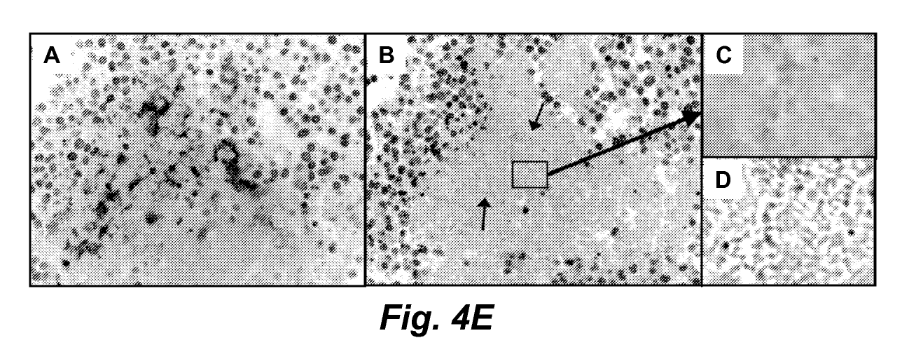

The second group of mice consisted of replicates injected with a single number of cells (2×10¹⁰ cells in 30 μl). Two tumor xenografts were implanted in each mouse. One tumor in each animal served as a contralateral control (left tumor, FIGS. 4A-4C) injected with 30 μl MSGM only. The test tumors (right tumor in each animal, FIGS. 4A-4C) were injected intratumorally with 6×10⁸ Magnetospirillum magneticum AMB-1 cells in 30 μl of MSGM.

T1-weighted images showed that compared to the pre-injection controls, the signal intensity increased 1.43-fold immediately (FIG. 4A), 2.02-fold after one day (FIG. 4B), and 1.77-fold after six days (FIG. 4C) (n=4, p<0.05 except on day 6). Note that no changes were seen in signal intensities of the control tumors for 6 days (FIG. 4D, white columns). At the completion of the experiment, animals were sacrificed and their tumor sections stained with Prussian blue (for iron) and Gram stain (for bacteria). Tumors receiving the bacteria had sections with regions of blue (indicative of iron) coinciding with adjacent sections with spots of red/pink indicative of bacteria; control tumors showed no spots from either stain, as shown in FIG. 4E.

### Intravenously Administered Magnetospirillum Magneticum AMB-1 Cells Accumulate in Mouse Tumors and Produce MRI Positive Contrast.

To examine the biodistribution of intravenously injected bacteria in mice, Magnetospirillum magneticum AMB-1 cells were radiolabeled with ⁶⁴Cu-PTSM. This enabled their distribution to be followed by highly sensitive Positron Emission Tomography (PET). Groups of three mice were injected intravenously with radiolabeled bacteria (1×10⁹ cells) or ⁶⁴Cu-PTSM (negative control). A third group was injected intratumorally with labeled bacteria (positive control). PET images were obtained at eight times between 0.5 and 64 hrs post-injection.

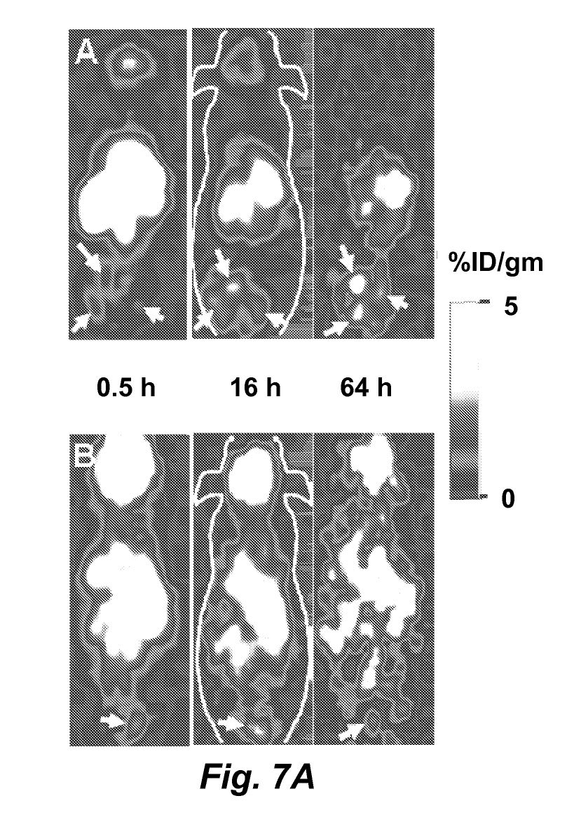

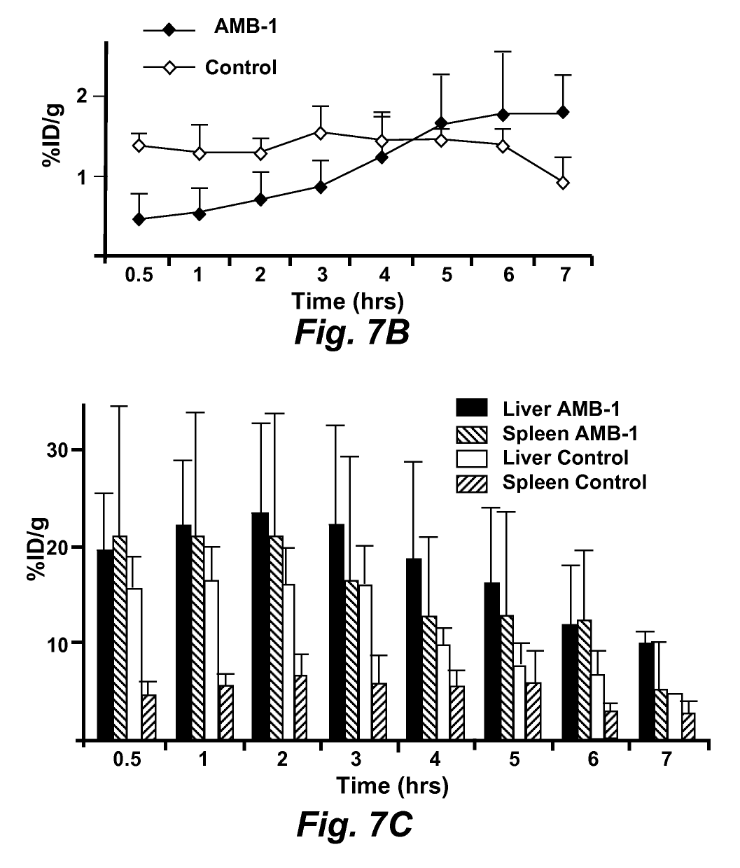

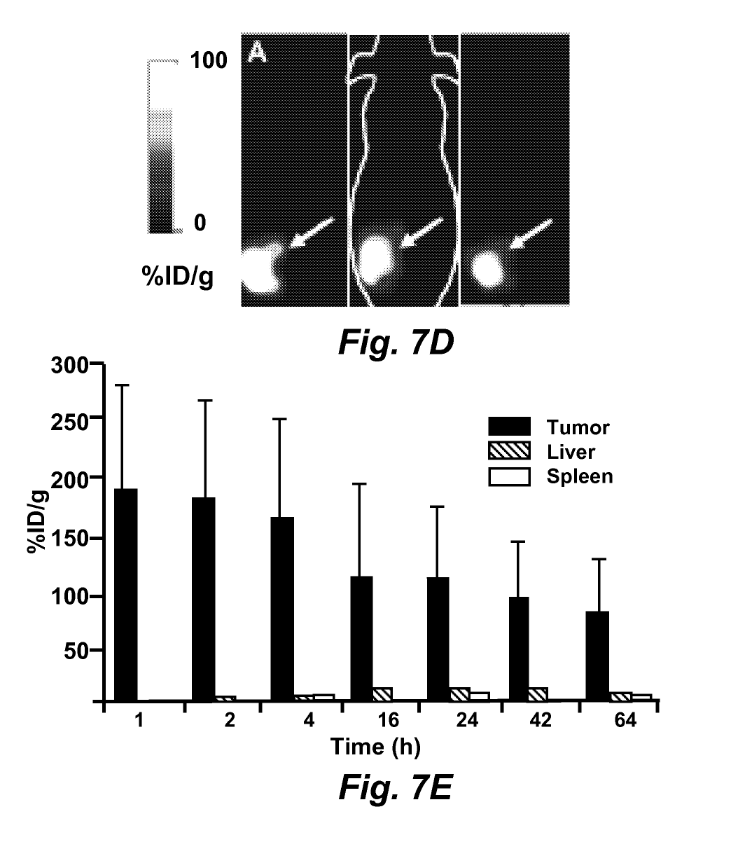

Shortly after intravenous injection (0.5 h), radioactivity (percent injected dose per gram of tissue, % ID/g) was found primarily in highly vascularized regions like liver and spleen (shown in FIG. 7A, upper panel). Little radioactivity was seen in the brain of animals injected with the radiolabeled bacteria, as opposed to the controls. While not wishing to be bound by any one theory, since bacteria cannot cross blood brain barrier, this difference indicates that ⁶⁴Cu-PTSM was largely retained in the labeled Magnetospirillum magneticum AMB-1 cells. The signal in the brains of the group injected with magnetotactic bacteria at 0.5 hr (1.2% ID/g) was 16.7% of that in the control group (7.2% ID/g), agreeing with in vitro ⁶⁴Cu-PTSM efflux from the bacteria of 17.6% after 0.5 hr

In the tumor, the PET signal up to the first 16 hours was higher in the control animals directly receiving ⁶⁴Cu-PTSM intravenously, compared with the test animals injected with ⁶⁴Cu-labeled bacteria, probably because of the porous nature of tumor vasculature. The tumor vasculature is expected to permit rapid diffusion of ⁶⁴Cu-PTSM into the tumor but slower penetration of micron-sized bacteria. The trend of increasing signal in the test tumors (FIG. 7B) had a higher signal after 64 hours compared with 0.5 hour (P=0.020). In control tumors, the signal began to decrease after 4 hours (FIG. 7B), which was also the case for normal tissue (liver and spleen) in both the control and test animals (FIG. 7C). This trend strongly indicated that the labeled bacteria accumulated in the tumor over the course of the experiment. After intratumoral injection (positive control group), the ⁶⁴Cu signal remained mainly confined to the tumor for 64 hrs, as shown in FIGS. 7D and 7E.

Because of the short half-life of ⁶⁴Cu (12.7 hours), a separate experiment with viable counts to investigate the distribution of AMB-1 for more than 64 hours was performed. Groups of three mice bearing 293T tumor xenografts were intravenously injected with 1×10⁹ Magnetospirillum magneticum AMB-1 through the tail vein. After 1, 3, and 6 days, groups of animals were sacrificed; the tumors, livers, and spleens were harvested, weighed, and homogenized; and samples were plated for colony forming units.

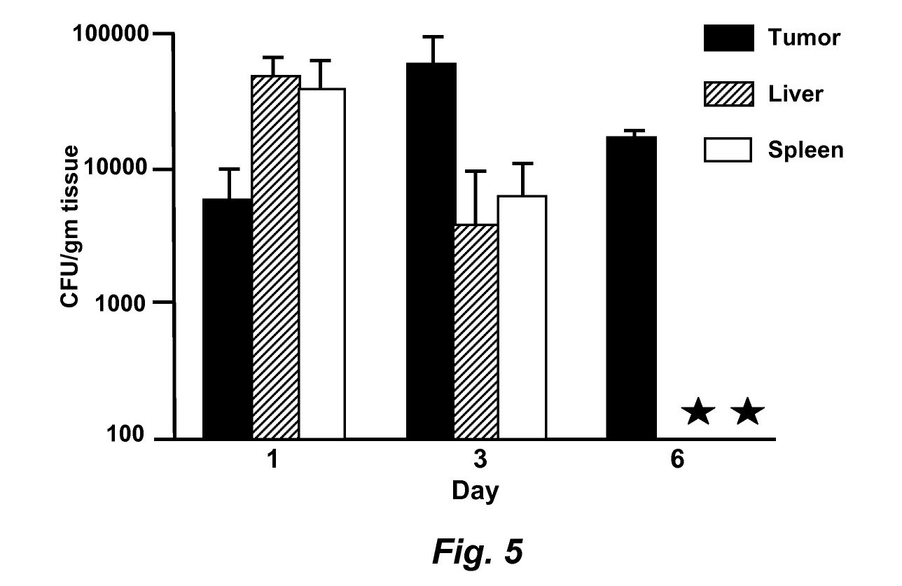

One day post-injection, the number of colony forming units recovered was higher in liver and spleen compared with the tumor. This trend reversed by day 3, and by day 6, no viable bacteria were found in liver or spleen; they were found only in tumor (as shown in FIG. 5).

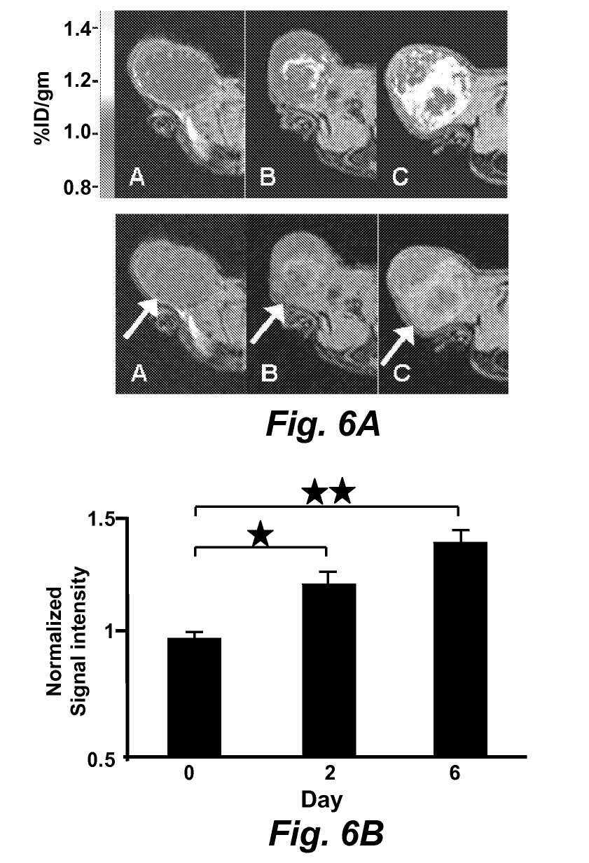

To determine the magnetic resonance imaging signal progression following intravenous AMB-1 administration, a group of four mice bearing 293T tumors were injected with low-Fe Magnetospirillum magneticum AMB-1 through the tail vein. T1-weighted images were collected before injection as well as 2 and 6 days post-injection. The signal increased 1.22-fold (P=0.003) after 2 days and 1.39-fold (P=0.0007) after 6 days, as shown in FIGS. 6A and 6B. Following the experiment, tumor sections stained with Prussian blue indicated the presence of iron for mice injected with Magnetospirillum magneticum AMB-1, but not for control animals. An independent experiment in which the images were acquired only on day 6 also showed 1.43 (±0.12)-fold (P=0.001; n=5) increase in signal compared with controls.

### Negative MRI Contrast Generation by Magnetospirillum Magneticum AMB-1 Cells.

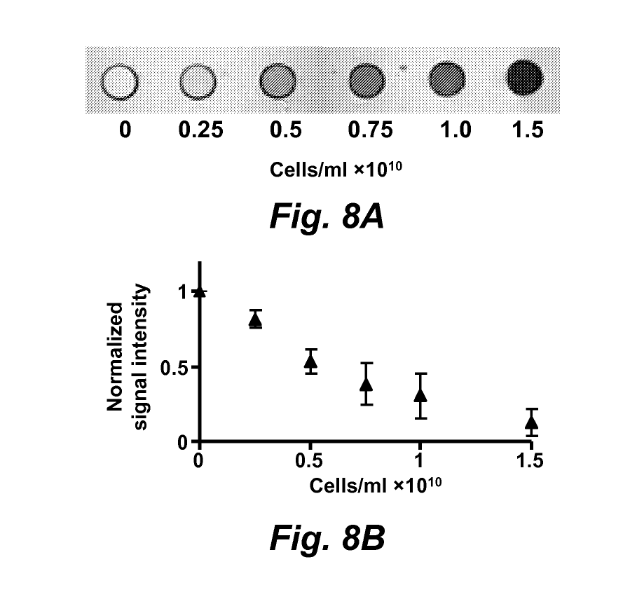

Magnetospirillum magneticum AMB-1 cells may also be cultivated in a medium where the iron source can be, but is not limited to, ferric malate. In this instance, the magnetite particles that form in the cells may be typically greater than about 50 nm. When such cells are then used in MRI imaging, the effect of the larger particles is to suppress the MRI signal, the signal intensity being inversely proportional to the concentration of the cells, as is shown in FIGS. 8A and 8B. Accordingly, it is considered within the scope of the present disclosure for such particle-laden cells to provide a negative-contrasting agent whereby, if introduced into a host animal, the bacterial cells will be concentrated at, in or on a targeted tissue such as, but not limited to, a tumorous tissue, which can then distinguished from the surrounding tissue by a suppression of the intensity of the MRI-generated signal.

One aspect of the present disclosure, therefore, provides methods of cultivating a magnetotactic bacterium, comprising: obtaining an isolated strain of magnetotactic bacteria capable of forming magnetite; and cultivating the magnetotactic bacteria in a growth medium comprising an iron salt, whereupon the cultivated magnetotactic bacteria synthesize magnetite particles having a diametric size between about 5 nm to about 50 nm, and where the cultivated magnetotactic bacteria are characterized as providing contrast enhancement of an magnetic resonance image of a cancerous lesion when contacted with said lesion.

In some embodiments of this aspect of the disclosure, the magnetotactic bacterium can be a strain of Magnetospirillum magneticum. In certain of these embodiments, the magnetotactic bacterium can be Magnetospirillum magneticum AMB-1 (ATCC Accession No. 700264).

In embodiments of the methods of this aspect of the disclosure, the iron salt can be selected from the group consisting of: iron malate, iron oxalate, iron succinate, iron citrate, iron chloride, iron sulfate, and iron nitrate, and wherein the iron is either ferric iron or ferrous iron.

In some embodiments, the iron salt can be ferric chloride, and the cultivated magnetotactic bacteria can have magnetite particles of about 10 to about 30 nm, and the magnetotactic bacteria are characterized as providing positive contrast enhancement of an magnetic resonance image thereof.

In other embodiments of this aspect, the cultivated magnetotactic bacteria can have magnetite particles of about 30 to about 60 nm, and the magnetotactic bacteria are characterized as proving negative contrast enhancement of an magnetic resonance image thereof.

Another aspect of the present disclosure provides a bacterial population cultivated in a growth medium comprising an iron salt, whereupon the cultivated bacteria comprises magnetite particles having a diametric size between about 5 nm to about 50 nm, and where the bacteria population is characterized as providing contrast enhancement of an magnetic resonance image of a cancerous lesion when contacted with said lesion.

In some embodiments of this aspect of the disclosure, the magnetotactic bacterium is a strain of Magnetospirillum magneticum.

In certain embodiments, the magnetotactic bacterium is Magnetospirillum magneticum AMB-1 (ATCC Accession No. 700264).

In some embodiments of this aspect of the disclosure, the cultivated magnetotactic bacteria can have magnetite particles of about 10 to about 30 nm, and the magnetotactic bacteria are characterized as proving positive contrast enhancement of an magnetic resonance image thereof.

In other embodiments of this aspect, the cultivated magnetotactic bacteria can have magnetite particles of about 30 to about 60 nm, and the magnetotactic bacteria are characterized as proving negative contrast enhancement of an magnetic resonance image thereof.

Another aspect of the disclosure provides compositions comprising magnetotactic bacteria cultivated in a growth medium comprising an iron salt, whereupon the cultivated bacteria comprise magnetite particles having a diametric size between about 5 nm to about 50 nm; and a pharmaceutically acceptable carrier, and where the cultivated magnetotactic bacteria are characterized as providing contrast enhancement of an magnetic resonance image of a cancerous lesion when contacted with said lesion.

In certain embodiments of this aspect of the disclosure, the magnetotactic bacteria can be Magnetospirillum magneticum AMB-1 (ATCC Accession No. 700264).

In certain embodiments of this aspect of the disclosure, the cultivated magnetotactic bacteria can have magnetite particles of about 10 to about 30 nm, and the magnetotactic bacteria are characterized as proving positive contrast enhancement of an magnetic resonance image thereof.

In other embodiments of this aspect of the disclosure, the cultivated magnetotactic bacteria have magnetite particles of about 30 to about 60 nm, and the magnetotactic bacteria are characterized as proving negative contrast enhancement of an magnetic resonance image thereof.

Yet another aspect of the disclosure provides methods of obtaining enhancement of positive contrast of a magnetic resonance image, comprising: delivering to a subject an amount of a composition comprising magnetotactic bacteria obtained by cultivating the magnetotactic bacteria in a growth medium comprising an iron salt, whereupon the cultivated bacteria comprise magnetite particles having a diametric size between about 5 nm to about 50 nm and where the cultivated magnetotactic bacteria are characterized as providing contrast enhancement of an magnetic resonance image of a cancerous lesion when contacted with said lesion; and a pharmaceutically acceptable carrier; allowing the magnetotactic bacteria to selectively target a tissue of the subject; and obtaining a magnetic resonance image of the subject, wherein the magnetotactic bacteria provide a magnetic resonance image having enhanced positive contrast.

In embodiments of this aspect of the disclosure, the magnetotactic bacteria can be Magnetospirillum magneticum AMB-1 (ATCC Accession No. 700264).

In certain embodiments of the method, the iron salt is ferric chloride.

In embodiments of this aspect, the composition can be delivered to a tissue of the subject, the tissue having, or suspected of having, a tumor therein, and where the magnetotactic bacteria provide a magnetic resonance image having enhanced positive contrast, wherein the image is of a tumor or cancerous lesion in the tissue of the subject.

In some embodiments, the composition can be delivered to a tumor or cancerous lesion in a tissue of the subject by intratumoral injection.

In other embodiments, the composition is delivered to a tumor in a tissue of the subject by intravenously administering the composition to the subject, whereupon the bacteria of the composition selectively target a tumor.

Yet another aspect of the disclosure provides methods of detecting a target tissue in a subject, comprising: delivering to a subject an amount of a composition comprising magnetotactic Magnetospirillum magneticum AMB-1 (ATCC Accession No. 700264) bacteria obtained by cultivating the magnetotactic bacteria in a growth medium comprising ferric chloride, whereupon the cultivated bacteria comprise magnetite particles having a diametric size between about 15 nm to about 30 nm, where the cultivated magnetotactic bacteria are characterized as providing contrast enhancement of an magnetic resonance image of a cancerous lesion when contacted with said lesion; and a pharmaceutically acceptable carrier, wherein the composition is delivered to a tissue of the subject; and obtaining a magnetic resonance image of the subject, where the magnetotactic bacteria provide a magnetic resonance image having enhanced positive contrast, thereby detecting the tissue of the subject.

In some embodiments of this aspect of the disclosure, the target tissue is a tumorous tissue, and wherein the composition is delivered to the tumorous tissue of the subject by intratumoral injection.

In some embodiments of this aspect of the disclosure, the composition is delivered to the tumorous tissue of the subject by intravenously administering the composition to the subject, whereupon the bacteria of the composition are selectively concentrated in a tumor.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### EXAMPLES

### Example 1

### Bacterial Strains and Growth Conditions

Magnetospirillum magneticum AMB-1 (ATCC Accession No. 700264) was used. The bacteria were grown at 30° C. with modified Magnetospirillum growth medium (MGSM) without supplemental iron (MSGM) as described in Komeili et al., Proc. Natl. Acad. Sci. U.S.A. (2004) 101: 3839-3844, incorporated herein by reference in its entirety. Cultures were grown in sealed tubes with 7% headspace of air. Bacterial cell density was determined by optical density (OD₆₀₀) measurements (Shimadzu BioSpec-1601 spectrophotometer) correlated to a standard curve. Iron (40 μM) was supplied either as ferric malate or ferric chloride.

### Example 2

### Magnetic Moment Measurements

Magnetospirillum magneticum AMB-1 cells were washed three times and suspended in MSGM at a range of cell densities. Their magnetic moment was measured with a Princeton MICROMAG 2900™ alternating gradient magnetometer by applying fields of ±5,000 Oersteds (Oe) in 200e steps. Magnetite content per bacterial cell was calculated from the magnetic moment (480 emu/cc) and density (5.2 g/cc) of magnetite. As 99.5% of iron consumed by magnetotactic bacteria is incorporated into magnetite (Grunberg et al., Appl. Environ. Microbiol. (2001) 70: 1040-1050), all cellular iron was assumed to be magnetite.

### Example 3

### MRI

For in vitro (phantom) studies, Magnetospirillum magneticum AMB-1 samples were washed twice and suspended in 3% gelatin (Sigma G9382) in plastic tubes. FERIDEX I.V.™ (Advanced Magnetics, Inc. Cambridge, Mass.) phantoms were prepared similarly. The phantoms aligned inside a 50 ml screw-cap tube that was subsequently filled with 0.7% agar. The gelatin was snap-solidified (4° C.) to maintain a homogenous cellular distribution.

For in vivo studies, female athymic nu⁻/nu⁻ mice (age, 6-8 weeks; Charles River) were used. Subcutaneous tumors were initiated by injecting 3×10⁶ 293T human embryonic immortalized kidney cells that produces firm tumors, permitting intratumoral injection. Palpable tumors formed within about two weeks.

Twice washed Magnetospirillum magneticum AMB-1 cells suspended in MSGM were injected either intratumorally, or intravenously by tail vein injection. For MR imaging, animals were anesthetized (isoflurane (2%) plus oxygen (1 l/min) delivered through a nose cone). They were kept warm (heated saline bags), their eyes were kept moist, and their respiration rate was measured every 15 min.

For all magnetic resonance measurements, a GE 3T MR scanner equipped with self-shielded gradients (40 mT/m, 150 mT/m/ms) was used. A custom-made radiofrequency (RF) quadrature coil was used for both RF excitation and signal reception (Ø=44 mm for in vivo and Ø=64 mm for in vitro). A 3D SPGR sequence (TE/TR=4/27 ms) with axial slice orientation was used to acquire T1-weighted images over 15 min (nominal resolution, 0.25×0.25×0.5 mm³).

For T1 measurements, an inversion-recovery fast spin echo (IR-FSE) sequence (TE/TR=8.2/10000 ms, FOV=64 mm, 128×128 matrix, 6-mm slice thickness) with inversion times (TI) of 50, 100, 150, 200, 400, 800, 1500, 2500, and 4000 ms was used. T1 values were estimated by a non-linear least squares fit of the data to a modified IR curve. A fitting parameter was used to account for the imperfect inversion along the z-axis caused by flip angle deviations due to B₁ inhomogeneities (Rakow-Penner at al., (2006) J. Magn. Reson. Imaging 23: 87-91, incorporated herein by reference in its entirety).

For T2 measurements an SE sequence (TR=10000 ms, FOV=64 mm, 128×128 matrix, 6-mm slice thickness) was used with TEs of 10, 15, 20, 40, 60, 100, 150, 200, 250, and 400 ms. T2 values were estimated by fitting the data to a mono-exponential decay curve. Relaxation rate constants (r₁=1/T1 and r₂=1/T2) were plotted versus the concentration of iron of the Magnetospirillum magneticum AMB-1, and the slope was used to determine relaxivity.

Signal intensity was measured from axial-slice 16-bit images using ImageJ (1.39 u with the Dicom input/output Plug-in, NIH freeware), background corrected and normalized to pre-injection values. Signal intensities were averaged among 5 consecutive axial-slice images, using mean values from ROIs drawn on the in vitro images and maximum values from the in vivo images. Maximum values were used for in vivo images because of the need to arbitrarily choose ROIs due to localized tumor colonization by Magnetospirillum magneticum AMB-1 following intratumoral or intravenous injection. Among the maximum values from five consecutive images, the standard deviation was consistently <10% of the mean.

### Example 4

### MicroPET Imaging

⁶⁴Cu was produced by cyclotron irradiation of an enriched ⁶⁴Ni target (Avila-Rodriguez et al., (2007) Appl. Radiat. Isot. 65: 1115-1120, incorporated herein by reference in its entirety), the ⁶⁴Cu-pyruvaldehyde-bis(N⁴-methylthiosemicarbazone) (⁶⁴Cu-PTSM) was prepared according to the method of Blower et al., Nucl. Med. Biol. 23: 957-980, incorporated herein by reference in its entirety; and Magnetospirillum magneticum AMB-1 cells were radio-labeled with ⁶⁴Cu-PTSM according to the method of Adonai et al., (2002) Proc. Natl. Acad. Sci. U.S.A. 99: 3030-3035, incorporated herein by reference in its entirety.

To optimize labeling conditions, the uptake and efflux of ⁶⁴Cu-PTSM was examined. For uptake, cells were incubated with 33 μCi for 0.5, 1, 2, 4, and 18 h. At each time point, triplicate samples were washed twice and activity was counted with a gamma counter. After 2 h, cells had taken up 56.7±2.4% activity, which increased only minimally by 4 hrs (57.4±2.0%); thus, 2 hrs incubation was chosen for labeling.

For efflux, cells were incubated with 123 μCi for 18 hrs then resuspended in ice-cold PBS. At 0, 0.5, 2, 4, and 24 h, triplicate samples were pelleted, the supernatant was aspirated, and the activity of the pellet was counted; 24 hrs later, the samples were found to retain 74.4±2.3% activity. Lack of toxicity of ⁶⁴Cu-PTSM to Magnetospirillum magneticum AMB-1 cells was verified after 24 hrs of incubation by microscopic observation of motile cells and by staining with the LIVE/DEAD™ BACLIGHT™ viability stain (Molecular Probes, Eugene, Oreg.).

For the in vivo experiment, Magnetospirillum magneticum AMB-1 cells were labeled with ⁶⁴Cu-PTSM by co-incubation for 2 h. 1×10⁹ Magnetospirillum magneticum AMB-1 cells suspended in 100 μl (approximately 220 μCi of activity) were injected intravenously via the tail-vein to three mice and intratumorally to a second group of three mice (positive controls). A third group of mice was injected intravenously with 100 μl of ⁶⁴Cu-PTSM alone (negative controls). The mice were anesthetized (as described above) and imaged at 0.5, 1, 2, 4, 16, 24, 42, and 64 hrs post-injection with a Siemens/Concorde Microsystems MicroPET rodent R4. The images were collected with static scans of 3 mins (at 0.5 h, 1 h, 2 h, and 4 h), 5 mins (at 16 h and 24 h), or 10 mins (at 42 h and 64 h). The microPET images were analyzed using ASIPRO VM™ 6.6.2.0 (Acquisition Sinogram Image PROcessing using IDL's Virtual Machine). ROIs were drawn on decay-corrected whole-body coronal images, and converted to % injected dose per gram of tissue (% ID/g) according to the method of Adonai et al., (2002) Proc. Natl. Acad. Sci. U.S.A. 99: 3030-3035, incorporated herein by reference in its entirety.

### Example 5

### Electron Microscopy and Size Analysis of Magnetite Particles

Suspensions of Magnetospirillum magneticum AMB-1 were fixed with 2.5% glutaraldehyde in 0.1 M phosphate buffer (pH 7.0) for 1 hr, then washed twice with wash buffer for 10 min. Post-fixation was performed with 1% osmium tetroxide in fixative buffer for 1 hrs and rinsed twice with double distilled water. The samples were left on 1% uranyl acetate in 20% acetone for 30 mins and dehydrated with a graded acetone series. Samples were then infiltrated and embedded in Spurr's resin.

Ultrathin sections were cut with a diamond knife and mounted onto uncoated copper grids. The sections were post-stained with 2% uranyl acetate for 15 min and 1% lead citrate for 5 min. The samples were examined with a CM-12 Phillips electron microscope. Magnetite particle diameter was measured for more than 100 magnetite particles per group from digitized TEM micrographs using ImageJ 1.39 u. The particle diameter histogram was made with 5 nm bins using MATLAB (The Mathworks, Natick, Mass.).

### Example 6

### Histology Preparation

Tumors were harvested from sacrificed animals and fixed in 10% buffered formalin overnight. Slices of 5 mm thickness were embedded in paraffin and longitudinally cut into sections of 5 μm thickness. Neighboring sections were stained with Perl's Prussian blue (for visualizing iron) and Gram stain (for visualizing bacteria), respectively.

### Example 7

### Viable Plate Counts

Nude mice bearing 293T subcutaneous tumor xenografts were injected with 1×10⁹ Magnetospirillum magneticum AMB-1 in 100 μl medium via the tail vein. Groups of three animals were sacrificed 1, 3, and 6 days after injection, and the tumor, liver, spleen and lungs were asceptically removed from each animal. The samples were rinsed with sterile phosphate buffered saline, weighed and homogenized, then centrifuged at 1000 rpm for 5 min. Samples from the supernatant were diluted, suspended in 5 ml of warmed MSGM with 0.7% agar, and plated on MSGM plates in duplicate. The plates were incubated in bags flushed with nitrogen gas at 30° C. for two weeks. Colony forming units (CFUs) were counted and normalized by tissue mass.

### Example 8

### Statistical Analysis

Two-tailed unpaired t-tests were performed for in vitro comparisons, and paired tests were used to compare contrast differences between experimental and control tumors; in paired t-tests, each tumor was compared to its contralateral control (intratumoral group) or to its own pre-injection value (intravenous. group). The Mann-Whitney significance test was used to evaluate the difference between distributions (of magnetite particle size or particle number). Statistical significance was determined by p<0.05.

1-15. (canceled)

16. A method of obtaining enhancement of positive contrast of a magnetic resonance image, comprising:
delivering to a subject an amount of a composition comprising magnetotactic bacteria obtained by cultivating the magnetotactic bacteria in a growth medium comprising an iron salt, whereupon the cultivated bacteria comprise magnetite particles having a diametric size between about 5 nm to about 50 nm, wherein the cultivated magnetotactic bacteria are characterized as providing contrast enhancement of an magnetic resonance image of a cancerous lesion when contacted with said lesion; and a pharmaceutically acceptable carrier;
allowing the magnetotactic bacteria to selectively target a tissue of the subject; and
obtaining a magnetic resonance image of the subject, wherein the magnetotactic bacteria provide a magnetic resonance image having enhanced positive contrast.

17. The method of claim 16, wherein the magnetotactic bacteria are Magnetospirillum magneticum AMB-1 (ATCC Accession No. 700264).

18. The method of claim 16, wherein the iron salt is ferric chloride.

19. The method of claim 16, wherein the composition is delivered to a tissue of the subject, the tissue having, or suspected of having, a tumor therein, and wherein the magnetotactic bacteria provide a magnetic resonance image having enhanced positive contrast, wherein the image is of a tumor in the tissue of the subject.

20. The method of claim 16, wherein the composition is delivered to a tumor in a tissue of the subject by intratumoral injection.

21. The method of claim 16, wherein the composition is delivered to a tumor in a tissue of the subject by intravenously administering the composition to the subject, whereupon the bacteria of the composition selectively target a tumor.

22. A method of detecting a target tissue in a subject, comprising:
delivering to a subject an amount of a composition comprising magnetotactic Magnetospirillum magneticum AMB-1 (ATCC Accession No. 700264) bacteria obtained by cultivating the magnetotatic bacteria in a growth medium comprising ferric chloride, whereupon the cultivated bacteria comprise magnetite particles having a diametric size between about 15 nm to about 30 nm, wherein the cultivated magnetotactic bacteria are characterized as providing contrast enhancement of an magnetic resonance image of a cancerous lesion when contacted with said lesion; and a pharmaceutically acceptable carrier, wherein the composition is delivered to a tissue of the subject; and
obtaining a magnetic resonance image of the subject, wherein the magnetotactic bacteria provide a magnetic resonance image having enhanced positive contrast, thereby detecting the tissue of the subject.

23. The method of claim 22, wherein the target tissue is a tumorous tissue, and wherein the composition is delivered to the tumorous tissue of the subject by intratumoral injection.

24. The method of claim 22, wherein the composition is delivered to the tumorous tissue of the subject by intravenously administering the composition to the subject, whereupon the bacteria of the composition are selectively concentrated in a tumor.
